SEQUENCE LISTING (13 sequences)

SEQ ID NO: 1 - protein, 1338 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLFIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 2 - protein, 687 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRGEHCNKKAVFSRISKFKSTRNDCTTQSNVKH

SEQ ID NO: 3 - protein, 733 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPELYTSTSPSSSSSSPLSSSSSSSSSSSS

SEQ ID NO: 4 - protein, 541 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKLPPANSSFMLPPTSFSSNYFHFLP

SEQ ID NO: 5 - protein, 665 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRGSEEMLLPK

SEQ ID NO: 6 - protein, 85 aa, Homo sapines
MLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 7 - protein, 433 aa, Homo sapiens
MVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 8 - protein, 361 aa, Homo sapiens
MNSDLLVDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 9 - protein, 85 aa, Homo sapiens; identical to SEQ ID NO: 6

SEQ ID NO: 10 - protein, 169 aa, Homo sapiens
MPVMRLFPCFLQLLAGLALPAVPPQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERRRPKGRGKRRREKQRPTDCHLCGDAVPRR

SEQ ID NO: 11 - protein, 221 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRHSPGRQSPDMPGDFRADAPSFLPPRRSLPMLFRMEWGCALTGSQSAVWPSSPVPEEIPRMHPGRNGKKQQRKPLREKMKPERCGDAVPRR

SEQ ID NO: 12 - protein, 149 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERCGDAVPRR

SEQ ID NO: 13 - protein, 170 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERRRPKGRGKRRREKQRPTDCHLCGDAVPRR

(19) United States
(12) Patent Application Publication
(10) Pub. No.: US 2024/0201207 A1
(43) Pub. Date: Jun. 20, 2024

(54) METHOD AND KIT FOR PREDICTING PREECLAMPSIA

(71) Applicant: MAX-DELBRÜCK-CENTRUM FÜR MOLEKULARE MEDIZIN IN DER HELMHOLTZ-GEMEINSCHAFT, Berlin (DE)

(72) Inventors: Florian Herse, Berlin (DE); Louise Bjorkholt Andersen, Kongens Lyngby (DK); Andreas Busjahn, Panketal (DE); Ralf Dechend, Biesenthal (DE)

(21) Appl. No.: 18/287,532

(22) PCT Filed: Apr. 19, 2022
(86) PCT No.: PCT/EP2022/060286

§ 371 Date: Oct. 19, 2023

(30) Foreign Application Priority Data
Apr. 19, 2021 (EP) 21169229.8

(51) Int. Cl. G01N 33/92

(52) U.S. Cl. CPC G01N 33/92; G01N 2333/475; G01N 2333/912; G01N 2800/368; G01N 2800/50

(57) ABSTRACT

The invention pertains to a method for the prediction of preeclampsia in a pregnant female human subject until the end of gestational week 16 of pregnancy based on metabolites from a blood sample obtained from said subject. According to the invention, the method comprises the following steps:
measuring the level of the following metabolites in the sample: 14,15-DHET, 13-HODE, 10,11-EDP, 21-HDHA, and 11,12-DHET, and
concluding based on the measured level whether the subject is likely to develop preeclampsia.

The invention relates to a method for the prediction of preeclampsia in a pregnant female human subject until the end of gestational week 16 of pregnancy based on metabolites (markers) from a blood sample obtained from said subject.

### DESCRIPTION

Preeclampsia is a major cause of morbidity and mortality during pregnancy.

The preeclampsia syndrome features new-onset hypertension (>140/90 mm Hg) after gestational week 20 accompanied by proteinuria (>300 mg/24 hours).

Overall, 5 to 10% of all pregnancies worldwide develop preeclampsia, a leading cause of morbidity and mortality in mother and child.

The object of the present invention is to provide a means for predicting the likelihood of a pregnant women to suffer from preeclampsia.

The present invention provides methods and kits for predicting and screening for preeclampsia. Particularly, the invention provides for preparing metabolite profiles of subject's blood samples, the metabolite profiles being indicative of the presence or absence of the development of preeclampsia during pregnancy. The invention further provides for evaluating the subject metabolite profiles for the presence or absence of one or more metabolite signatures that are indicative of preeclampsia.

The inventors have surprisingly found that certain lipids, namely eicosanoids, signaling molecules derived from polyunsaturated fatty acids which exert complex control over several communication pathways in the human body, can be used to predict preeclampsia. They found that measuring the level of certain metabolites in the blood sample allows for the prediction of the presence or absence of preeclampsia with high predictive values.

Here, the term “eicosanoids” is used to include oxylipins that are derived from arachidonic acids (AA), linoleic acid (LA), or eicosapentaenoic- and docosahexaenoic-acid (EPA/DHA).

In other words, measuring these metabolite is necessary and sufficient for the prediction whether a pregnant female human subject will develop preeclampsia or not based on a blood sample obtained from said subject by measuring the level of metabolites in the sample. that are chosen from the metabolites listed herein, in particular from the metabolites in table 1, and concluding based on the measured levels whether the subject will develop preeclampsia or not.

In one aspect, the invention provides a method for preparing metabolite profiles that are indicative of the presence or absence of preeclampsia. The metabolite profiles are prepared from subject blood samples. The number of metabolites in the metabolite profile may be selected so as to offer a convenient and cost-effective means for screening samples for the future presence or absence of preeclampsia with high sensitivity and high specificity. Generally, the metabolite profile includes the amount or the concentration (referred to here as “level”) of from 5 to about 21 metabolites or more. The concentration can be inferred from the measured amount since the volume used in the method is known.

In a second aspect, the invention provides a method for predicting preeclampsia. In this aspect the method comprises preparing a metabolite profile by measuring the level of at least 5 metabolites in a subject's blood sample. where the level of such metabolites are indicative of the presence or absence of preeclampsia. The metabolites may be the ones listed in table 1, and exemplary sets of such profiles are disclosed herein, for example, in the figures. The method further comprises evaluating the profile for the presence or absence of a metabolite signature indicative of preeclampsia, to thereby conclude whether the subject will or will not develop preeclampsia.

Specifically, the method of the invention comprises at least the following steps: measuring the abundance of at least 5 metabolites in the sample that are chosen from the metabolites listed in table 1, and concluding, based on the measured levels, whether the subject has preeclampsia or not. Measuring the level of metabolites may comprise preparing serum or plasma from blood samples, and measuring levels of the metabolites using mass spectrometry. Alternatively, other methods for determining metabolite levels may be employed.

More specifically, the invention refers in one aspect to a method for the prediction of preeclampsia in a pregnant female human subject until the end of gestational week 16, preferably within the first trimester of pregnancy based on metabolites from a blood sample obtained from said subject. For example, the method of the invention may be practiced at gestational week 12. The method allows for a classification of subjects into healthy women and preeclamptic women.

The term “until the end of gestational week 16” refers to the time of pregnancy from gestational week 1 to the end of gestational week 16 of pregnancy. In certain embodiments, the method is used in the first trimester of pregnancy, i.e. from gestational week 1 to the end of gestational week 12 of pregnancy. The method may also be used from gestational week 4, 5, or 6 to gestational week 12, 13, 14, 15 or 16.

In one embodiment, the method comprises the following steps:
measuring the level of each of the following metabolites in the sample: 14,15-DHET, 13-HODE, 10,11-EDP, 21-HDHA, and 11,12-DHET, and
concluding based on the measured level whether the subject is likely to develop preeclampsia.

The metabolites 14,15-DHET, 13-HODE, 10,11-EDP, 21-HDHA, and 11,12-DHET are listed in table 1.

The identities and/or combinations of metabolites that make up or are included in expression profiles are disclosed in table 1. In particular embodiments, measuring the level of metabolites listed in table 2 and/or table 3 may be included in the method.

TABLE 1
Group of 5 Metabolite Markers

| Abbreviation | Chemical Name |
|---|---|
| 14,15-DHET | 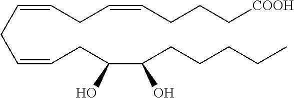 [SMILES: CCCCC[C@@H](O)[C@@H](O)C/C=C\C/C=C\C/C=C\CCCC(=O)O] |
| | (±)14,15-dihydroxy-5Z,8Z,11Z-eicosatrienoic acid |
| 11-HETE | 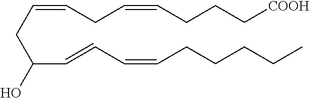 [SMILES: CCCCC/C=C\C=C\C(O)C/C=C\C/C=C\CCCC(=O)O] |
| | (±)11-HETE |
| | CAS: 73804-65-6 |
| 10,11-EDP | 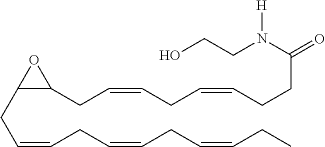 [SMILES: CC/C=C\C/C=C\C/C=C\CC1OC1C/C=C\C/C=C\CCC(=O)NCCO] |
| | N-(2-hydroxyethyl)-9-[3-(2Z,5Z,8Z)-2,5,8-undecatrien- |
| | 1-yl-2-oxiranyl]-4Z,7Z-nonadienamide |
| | CAS: 2123484-71-7 |
| 13-HODE | 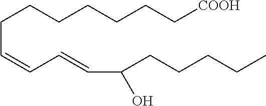 [SMILES: CCCCCC(O)/C=C/C=C\CCCCCCCC(=O)O] |
| | (±)-13-hydroxy-9Z,11E-octadecadienoic acid, |
| | CAS: 73804-64-5 |
| 21-HDHA | 21-hydroxy-4Z,7Z,10Z,13Z,16Z,19Z-docosahexaenoic acid |

In certain embodiments of the method of the invention, the levels of additional markers to the five markers of table 1 are measured.

In certain embodiments of the method, the levels of at least one (1, 2, 3, or more) of the following metabolites are measured in the sample in addition to the metabolites of table 1: 7,8-EDP, 16,17-DiHDPA, 12,13-DiHOME, 11,12-DHET, 9,10-DiHOME, 5,6-DiHETE; 10-HDHA, 11,12-EET, 15-HETE, 7,8-DiHDPA, PGE2, 14,15-EET, 18-HETE, EODAcis, EODAtrans, and 13,14-DiHDPA. These markers are listed in table 2.

TABLE 2
Group of additional 16 Metabolite Markers

| Abbreviation | Chemical/Alternative Name |
|---|---|
| 7,8-EDP | 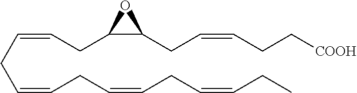 [SMILES: CC/C=C\C/C=C\C/C=C\C/C=C\C[C@H]1O[C@H]1C/C=C\CCC(=O)O] |
| | (±)-(4Z)-6-[3-(2Z,5Z,8Z,11Z)-2,5,8,11-tetradecatetraen-1-yl-2-oxiranyl]- |
| | 4-hexenoic acid |
| | CAS: 895127-66-9 |
| 16,17- DiHDPA | 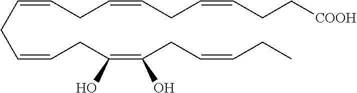 |
| | (±)16,17-dihydroxy-4Z,7Z,10Z,13Z,19Z-docosapentaenoic acid |
| | CAS: 1345275-27-5 |
| 12,13- DIHOME | 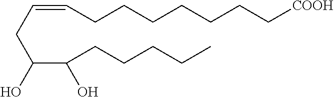 [SMILES: CCCCCC(O)C(O)C/C=C\CCCCCCCC(=O)O] |
| | 12,13-dihydroxy-9Z-octadecenoic acid |
| | CAS: 263399-35-5 |
| 11,12-DHET | 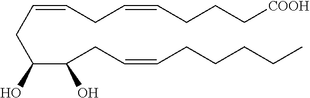 [SMILES: CCCCC/C=C\C[C@@H](O)[C@@H](O)C/C=C\C/C=C\CCCC(=O)O] |
| | (±)11,12-dihydroxy-5Z,8Z,14Z-eicosatrienoic acid |
| 9,10- DIHOME | 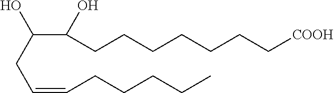 [SMILES: CCCCC/C=C\CC(O)C(O)CCCCCCCC(=O)O] |
| | CAS: 263399-34-4 |
| 5,6-DiHETE | 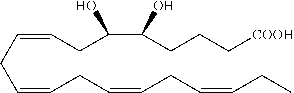 [SMILES: CC/C=C\C/C=C\C/C=C\C/C=C\C[C@@H](O)[C@@H](O)CCCC(=O)O] |
| | (±)5,6-dihydroxy-8Z,11Z,14Z,17Z-eicosatetraenoic acid |
| | CAS: 845673-97-4 |
| 10-HDHA | 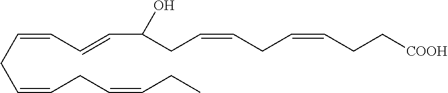 [SMILES: CC/C=C\C/C=C\C/C=C\C=C\C(O)C/C=C\C/C=C\CCC(=O)O] |
| | (±)10-hydroxy-4Z,7Z,11E,13Z,16Z,19Z-docosahexaenoic acid |
| | CAS: 90780-50-0 |
| 11,12-EET | 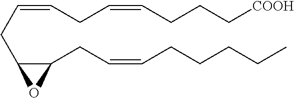 [SMILES: CCCCC/C=C\C[C@H]1O[C@H]1C/C=C\C/C=C\CCCC(=O)O] |
| | (±)11(12)- epoxy- 5Z, 8Z, 14Z- eicosatrienoic acid |
| | CAS: 81276-02-0 |
| 15-HETE | 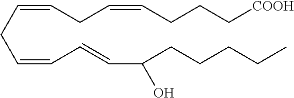 [SMILES: CCCCCC(O)/C=C/C=C\C/C=C\C/C=C\CCCC(=O)O] |
| | CAS: 73836-87-0 |
| 7,8-DiHDPA | 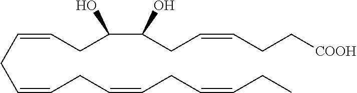 [SMILES: CC/C=C\C/C=C\C/C=C\C/C=C\C[C@@H](O)[C@@H](O)C/C=C\CCC(=O)O] |
| | (±)7,8-dihydroxydocosa-4Z,10Z,13Z,16Z,19Z-pentaenoic acid |
| | CAS: 168111-93-1 |
| PGE2 | 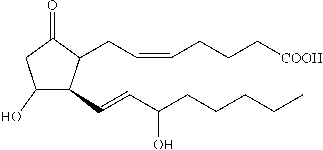 [SMILES: CCCCCC(O)/C=C/[C@H]1C(O)CC(=O)C1C/C=C\CCCC(=O)O] |
| | Prostaglandin E2 |
| | CAS: 363-24-6 |
| 14,15-EET | 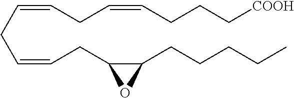 [SMILES: CCCCC[C@H]1O[C@H]1C/C=C\C/C=C\C/C=C\CCCC(=O)O] |
| | (±)14,15-DiHETrE |
| 18-HETE | 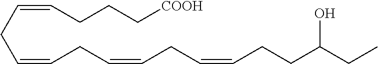 [SMILES: CCC(O)CC/C=C\C/C=C\C/C=C\C/C=C\CCCC(=O)O] |
| | (±)18-hydroxy-5Z,8Z,11Z,14Z-eicosatetraenoic acid |
| | CAS: 133268-58-3 |
| EODAcis | 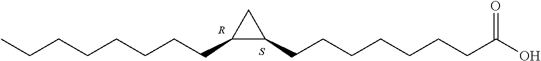 |
| | 9,10-epoxyoctadecanoic acid; (±)-cis-9,10-epoxyoctadecanoic acid |
| | CAS: 24560-98-3 |
| EODAtrans | 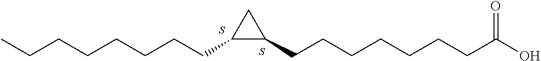 |
| | trans-9,10-epoxystearic acid; (±)-trans-9,10-epoxyoctadecanoic acid |
| | CAS: 13980-07-09 |
| 13,14- DiHDPA | 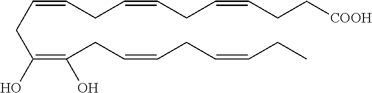 [SMILES: CC/C=C\C/C=C\C/C(O)=C(/O)C/C=C\C/C=C\C/C=C\CCC(=O)O] |
| | 13,14-dihydroxy-4Z,7Z,10Z,16Z,19Z-docosapentaenoic acid |
| | CAS: 1345275-24-2 |

In preferred embodiments of the method, the levels of five or more (6, 7, 8, . . . 16) of the metabolites of table 2 are measured in the sample in addition to the markers listed in table 1. In certain embodiments, a metabolite listed in table 3 is measured in addition to the metabolites listed in table 1 and, optionally, one or more of the metabolites listed in table 2.

TABLE 3
Group of additional Metabolite Markers

| Abbreviation | Chemical/Alternative Name |
|---|---|
| 11-HETE | 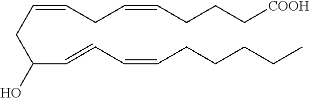 [SMILES: CCCCC/C=C\C=C\C(O)C/C=C\C/C=C\CCCC(=O)O] |
| | ((±)11-hydroxy-5Z,8Z,12E,14Z-eicosatetraenoic acid |
| | CAS: 73804-65-6 |
| 15-keto-PGE2 | 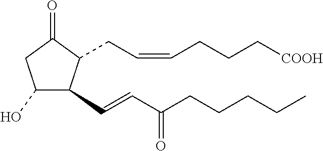 [SMILES: CCCCCC(=O)/C=C/[C@H]1[C@H](O)CC(=O)[C@@H]1C/C=C\CCCC(=O)O] |
| | 9,15-dioxo-11α-hydroxy-prosta-5Z,13E-dien-1-oic acid |
| | CAS: 26441-05-4 |
| 13,14-dihydro- 15-keto-PGE2 | 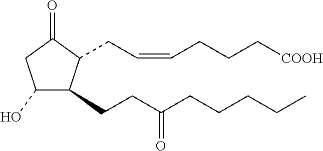 [SMILES: CCCCCC(=O)CC[C@H]1[C@H](O)CC(=O)[C@@H]1C/C=C\CCCC(=O)O] |
| | 9,15-dioxo-11α-hydroxy-prost-5Z-en-1-oic acid |
| | CAS : 363-23-5 |

In certain embodiments of the method of the invention, the level of the metabolites is measured simultaneously. In other embodiments, levels of metabolites are measured in a consecutive manner. In other embodiments, the level of certain the metabolites is measured simultaneously, i.e. in groups while groups are measured consecutively.

In certain embodiments of the method of the invention, additional metabolites are measured. Such additional metabolites may be any eicosanoid.

In certain embodiments of the method of the invention, the level of 11-HETE shown in table 3 is additionally measured in the sample in addition to the metabolites listed in table 2 and possibly other metabolites, such as other metabolites listed in table 3.

In certain embodiments of the method of the invention, additional metabolites are measured. Such additional metabolites may be any eicosanoid.

Preferred embodiments of the method of the invention are disclosed throughout the application, in particular in the figures.

In one preferred embodiment of the invention, the method comprises the following steps:
measuring the level of the metabolites listed in table 1 in the sample (14,15-DHET, 13-HODE, 10,11-EDP, 21-HDHA, and 11-HETE) together with the following selection of markers of table 2: 5,6-DiHETE; 11,12-DHET; 13,14-DiHDPA; 12,13-DiHOME; 9,10-DiHOME; 18-HETE, and
concluding based on the measured level whether the subject is likely to develop preeclampsia.

In another preferred embodiment, the method comprises the following steps:
measuring the level of the metabolites listed in table 1 in the sample (14,15-DHET, 13-HODE, 10,11-EDP, 21-HDHA, and 11-HETE) together with the following selection of markers of table 2: 5,6-DiHETE; 11,12-DHET; 13,14-DiHDPA; 12,13-DiHOME; 9,10-DiHOME; 18-HETE; 14,15-EET, and
concluding based on the measured level whether the subject is likely to develop preeclampsia.

In certain embodiments, the level of the angiogenic marker concentrations soluble fms-like tyrosine kinase 1 (sFlt-1) (SEQ ID NOs 1-9) and/or the marker placental growths factor (PlGF) (SEQ ID NOs 10-12) is measured in addition to the markers of table 1 and possibly additional markers, as described herein. This further increases the accuracy of the method of the present invention.

The level of these markers sFlt-1 und PlGF may be determined using mass spectrometry or a quantitative immune assay, as known in the art.

In certain embodiments, the method comprises the isolation of eicosanoids from a blood sample, in particular from serum or plasma. For the isolation, a separation column in the form of a solid-phase column may be used for extraction of the eicosanoids.

In certain embodiments of the method, the measuring of the metabolite level (and possibly other markers) is performed using a mass spectrometer. In particular, a mass spectrometer may be used in combination with reversed phase high-performance liquid chromatography (HPLC).

In particular embodiments, a triple quadrupole mass spectrometer (TQMS) may be used in the method of the invention, i.e. a tandem mass spectrometer comprising two quadrupole mass analyzers in series, with a radio frequency-only quadrupole between them to act as a cell for collision-induced dissociation, as known in the art.

The present invention provides an in-vitro diagnostic test system (IVD) that is trained, as described further below, for the prediction of preeclampsia. For example, in order to determine whether a subject has or will develop preeclampsia, reference metabolite level values for preeclampsia positive and negative samples are determined. The metabolites can be quantitatively measured on an adequate set of training samples comprising cases and controls, and with adequate clinical information on preeclampsia status, applying adequate quality control measures, and on an adequate set of test samples, for which the detection is yet to be made. With such quantitative values for the metabolites and the clinical data for the training samples, a classifier can be trained and applied to the test samples to calculate the probability of the future presence or non-presence of preeclampsia during pregnancy.

Various classification schemes are known for classifying samples between two or more classes or groups, and these include, without limitation: Naïve Bayes, Support Vector Machines, Nearest Neighbors, Decision Trees, Logistics, Artificial Neural Networks, and Rule-based schemes. In addition, the predictions from multiple models can be combined to generate an overall prediction. Thus, a classification algorithm or “class predictor” may be constructed to classify samples. The process for preparing a suitable class predictor is reviewed in R. Simon, Diagnostic and prognostic prediction using gene expression profiles in high-dimensional microarray data, British Journal of Cancer (2003) 89, 1599-1604.

In this context, the invention teaches an in-vitro diagnostic test system (IVD) that is trained in the prediction of a preeclampsia referred to above, comprising at least 5 metabolites, which can be quantitatively measured on an adequate set of training samples comprising cases and controls, with adequate clinical information on preeclampsia status, applying adequate quality control measures, and on an adequate set of test samples, for which the detection yet has to be made. Given the quantitative values for the metabolites and the clinical data for the training samples, a classifier can be trained and applied to the test samples to calculate the probability of the presence or absence of preeclampsia.

In the method of the invention, the level of several metabolite molecules is determined in a relative or an absolute manner, wherein an absolute measurement of metabolite level is preferred. The level is, if applicable, compared with that of other individuals, or with multivariate quantitative thresholds.

The conclusion whether the subject will develop preeclampsia or not is preferably reached on the basis of a classification algorithm, which can be developed using, for example, a random forest method, a support vector machine (SVM), or a K-nearest neighbor method (K-NN), such as a 3-nearest neighbor method (3-NN), as known in the art.

In certain embodiments of the method of the invention, the decision whether the subject has preeclampsia comprises the step of training a classification algorithm on a training set of cases and controls, and applying it to measured metabolite levels.

The determination of the level of the metabolites described herein is performed from blood samples using quantitative methods. For example, metabolite levels can be measured by mass spectrometry.

The blood sample used in the method of the invention may be, for example, a whole blood sample, a serum sample or a plasma sample. Metabolites are usually isolated by solid-phase column extraction from a certain volume of whole blood, serum, or plasma. In certain embodiments, serum or plasma samples are used. In certain embodiments, the metabolites are isolated by solid-phase column extraction plasma or serum.

In certain embodiments of the method of the invention, the method uses 5 or more markers, in particular 5, 6, 7, 8, 9, 10, 11, 12, 13, 14, 15, 16, 17, 18, 19, 20, 21 or more markers. In certain embodiments of the method, at least 5, at least 6, at least 7, at least 8, at least 9, at least 10, at least 11, at least 12, at least 13, at least 14, at least 15, at least 20, at least 25 or at least 30 markers are used.

Responsive to the measured level indicating that the subject is likely to develop preeclampsia, an anti-preeclampsia drug in an amount effective to inhibit or prevent preeclampsia may be administered.

When it is concluded in the method based on the measured levels of metabolites whether the subject is likely to develop preeclampsia, the subject may be treated by a physician. One option of treatment may be the administration of acetylsalicylic acid (ASA, Aspirin®) to the subject in an appropriate dose. For example, 100 mg ASA per day may be administered. Administration may be started before gestational week 16, but could also be administered later or earlier. Drugs may also include calcium supplementation, or drugs to treat hypertension.

In another aspect, the invention refers to the use of a method as described herein for the prediction of preeclampsia in a pregnant female human subject until the end of gestational week 16 of pregnancy based on metabolites from a blood sample, in particular from a plasma sample or serum sample.

The invention also refers to the use of a marker for determining the risk of preeclampsia in a pregnant woman in the first trimester of pregnancy, comprising measuring the concentration of at least the markers listed in table 1. Such a method is performed in vitro.

In certain aspects, the invention is directed to a kit for detecting the level of metabolites in the subject's blood sample. where this “profile” allows for the conclusion of whether the subject will develop preeclampsia or not. In another aspect, the invention relates to a standard that allows for the detection of the metabolites associated with preeclampsia, in particular those described herein.

In another aspect, the invention refers to a kit for the prediction of preeclampsia in a female human subject based on metabolites obtained from a blood sample, comprising means for measuring the level of at least the markers listed in table 1.

The kit may comprise or contain at least one compound of table 1 as a standard for use in mass spectrometry. The standard can be used to ensure quantitative reproducibility of the method of the invention.

In certain embodiments, the kit comprises or contains 1, 2, 3, 4, or 5 compounds of table 1 as a standard for use in mass spectrometry.

The kit may also comprise at least one compound of table 2 and/or table 3 as a standard for use in mass spectrometry.

The solution of the standard compound of table 1, 2 and/or 3 is usually prepared gravimetrically and is supplied in a deactivated glass ampule sealed under an inert gas, such as argon. The concentration is usually verified by comparison to an independently prepared calibration standard.

In certain embodiments, the kit may also comprise or contain at least one solid-phase column for isolating eicosanoids from blood, in particular from serum or plasma from blood samples.

In another aspect, the invention refers to the use of such a kit for the prediction of preeclampsia in a human subject based on metabolites from a blood sample, comprising means for measuring the level of at least the markers listed in table 1. The kit may be used in a method for predicting preeclampsia as described herein. The kit may contain instructions for treating the development of preeclampsia.

The kit may also comprise a chromatography column and/or a quality control specimen.

In certain embodiments, the standard is a marker as listed in table 1, table 2 and/or table 3 that is labeled to make it distinguishable from the marker of the sample that is to be measured, such that the standard can be measured simultaneously to the sample in a mass spectrometer. In certain embodiments, the standard is a marker as listed in table 1, table 2 and/or table 3 that is isotope labeled, in particular is a marker as listed in table 1, table 2 and/or table 3 that is isotope labeled.

In certain embodiments, the standard is deuterated, it contains one or more, in particular one deuterium atom instead of a hydrogen atom.

In another aspect, the invention refers to the use of a mass spectrometer for performing a method for the prediction of preeclampsia as described herein.

In another aspect, the invention refers to a method for preparing a metabolite profile that is indicative of the presence or absence of preeclampsia in a subject, comprising:
isolating metabolites from a blood sample obtained from the subject, and
measuring the level of the following metabolites in the sample: 14,15-DHET, 13-HODE, 10,11-EDP, 21-HDHA, and 11,12-DHET.

In yet another aspect, the invention refers the use of a set of markers for determining within the first trimester of pregnancy the risk for a pregnant woman to suffer from preeclampsia, comprising measuring the level of the following metabolites in the sample: 14,15-DHET, 13-HODE, 10,11-EDP, 21-HDHA, and 11,12-DHET.

Human protein sequences of sFlt-1 are disclosed as SEQ ID NO 1-9. Of this gene, 9 transcripts (splice variants), 198 orthologues, and 54 paralogues are known.

Human protein sequences of PIGF are disclosed as SEQ ID NO 10-13. Of this gene, 8 transcripts (splice variants), 225 orthologues and 4 paralogues are known.

### EXAMPLES

The inventors measured eighty-one free circulating eicosanoids derived from linoleic-, eicosapentaenoic-, docosahexaenoic- and arachidonic acids by Triple-Quad-Tandem mass spectrometry in serum collected at gestational week 12 in a cohort study (100 total, 50 cases, 50 controls). The preeclampsia phenotype was heterogeneous with predominantly mild, late-onset disease. Using a regression-tree approach, prediction models incorporated clinical data, angiogenic markers and eicosanoid metabolites, compared with a model including only standard clinical variables in combination with sFlt-1 and PlGF.

Here, the term “eicosanoids” is used to include oxylipins that are derived from arachidonic acids (AA), linoleic acid (LA), or eicosapentaenoic- and docosahexaenoic-acid (EPA/DHA). Metabolites are generated following phospholipase A2 (PLA2)-mediated release by cyclooxygenases (COX), lipoxygenases (LOX), cytochrome P450 (CYP) enzymes, or autoxidation. Dietary enrichment with EPA/DHA from fish and fish oil derivatives is known to promote cardio-protective effects.

A regression tree approach for predicting preeclampsia in the first trimester demonstrated good predictive value, in particular when combining it with clinical data, maternal blood pressure and sFlt-1/PlGF: sensitivity 56, specificity 94, AUC 78. However, the use of eicosanoid metabolites substantially increased the overall performance of a regression-tree approach, sensitivity 71, specificity 99, AUC 87.

### Materials and Methods

### Subjects and Characterization

The case-control study was nested in the prospective, population-based Odense Child Cohort, a cohort including newly pregnant women between Jan. 1^{st} 2010 and Dec. 31^{st} 2012 in Odense, Denmark. All participants gave written informed consent. The study complied with the Helsinki declaration and was approved by the Regional Scientific Ethical Committee for Southern Denmark (no. S-20090130).

Longitudinal serum samples from early and late pregnancy (median 12 weeks of gestation and 29 weeks of gestation) were included from 52 preeclamptic women and 51 healthy pregnancies, matched on gestational age at blood sampling, body mass index, parity, twinning pregnancies, and smoking status. The criteria for preeclampsia were repeated measures of hypertension (>140/90 mmHg) after 20 weeks of gestation with at least +1 for proteinuria on sterile urine dipstick. The more severe subtypes were further defined: early-onset PE (EOPE) with debut before 34+0 weeks of gestation, and preeclampsia with preterm delivery (PreEOPE) before 37+0 weeks of gestation.

### Eicosanoid Measurement

This HPLC/MS/MS based method (Lipidomix, Germany) comprised all COX, LOX, CYP and auto-oxidation generated free eicosanoids originated from the substrates AA, LA or EPA/DHA. Eicosanoids were isolated by Varian Bond Elute Certify II solid-phase column extraction from 350 μl serum. Extracts were analyzed by Triple-Quad-Tandem mass spectrometry Agilent 6460 (Agilent Technologies, Santa Clara, CA) coupled with a rapid resolution HPLC Agilent 1200.

### Angiogenic Factor Measurements

Measurements for BRAHMS sFLT-1 and PIGF KRYPTOR immune assays were performed on the fully automated KRYPTOR compact Plus system (KRYPTOR PIGF and KRYPTOR sFLT-1: Thermo Fisher Scientific) as described in Andersen et al., 2016.

### Statistical Methods

All quantitative variables were tested for Gaussian distribution. Descriptive statistics are mean and SD or median and 25^{th}/75^{th} percentile as appropriate. Group differences were tested by t-test or Wilcoxon-test. For time differences, pairwise tests were applied. Correlations between clinical parameters and metabolites were analyzed by Spearman rank correlations and plotted in correlation heat maps. To determine diagnostic values, we applied receiver operating characteristic (ROC)-curves and logistic regressions. As base model, sFLT-1 and PlGF were included. This model was then extended by each metabolite separately. Metabolites significantly improving model fit were then entered into the final multivariate model. Areas under the curve (AUC) of base vs. final model were compared by one-side significance test. Prediction of disease groups by logistic regression makes the assumption of a linear risk increase per unit of predictor and cannot determine threshold effects or U-shaped risks. Therefore, regression trees as a non-linear threshold-based classification approach were included. For this approach, no pre-selection of metabolites was applied. All analyses were conducted in R version 3.3.1 (R Core Team 2016: A language and environment for statistical computing R Foundation for Statistical Computing, Vienna, Austria: https://www.R-project.org/).

### FIGURES

FIG. 1 A Decision tree of 21 markers with hierarchical definition of the selected marker and cut-of value for step-wise optimization of classification. Lower boxes represent the final classification of the respective branch of the decision tree with assigned diagnosis, relative frequency of correct classification, and % of total population assigned to that branch.
B (top): Importance of biomarkers for the final classification; as each marker can be used more than once in the decision tree, this is not identical to the tree hierarchy. (bottom): ROC-plot for sensitivity and specificity for the continuous outcome probabilities given by the decision tree. The optimal cut-off is selected to optimize both by computing the maximum distance from the diagonal.

FIG. 2 A Decision tree of 20 markers with hierarchical definition of the selected marker and cut-of value for step-wise optimization of classification. Lower boxes represent the final classification of the respective branch of the decision tree with assigned diagnosis, relative frequency of correct classification, and % of total population assigned to that branch.
B (top): Importance of biomarkers for the final classification: as each marker can be used more than once in the decision tree, this is not identical to the tree hierarchy. (bottom): ROC-plot for sensitivity and specificity for the continuous outcome probabilities given by the decision tree. The optimal cut-off is selected to optimize both by computing the maximum distance from the diagonal.

FIG. 3 A Decision tree of 19 markers with hierarchical definition of the selected marker and cut-of value for step-wise optimization of classification. Lower boxes represent the final classification of the respective branch of the decision tree with assigned diagnosis, relative frequency of correct classification, and % of total population assigned to that branch.
B (top): Importance of biomarkers for the final classification; as each marker can be used more than once in the decision tree, this is not identical to the tree hierarchy. (bottom): ROC-plot for sensitivity and specificity for the continuous outcome probabilities given by the decision tree. The optimal cut-off is selected to optimize both by computing the maximum distance from the diagonal.

FIG. 4 A Decision tree of 18 markers with hierarchical definition of the selected marker and cut-of value for step-wise optimization of classification. Lower boxes represent the final classification of the respective branch of the decision tree with assigned diagnosis, relative frequency of correct classification, and % of total population assigned to that branch.
B (top): Importance of biomarkers for the final classification: as each marker can be used more than once in the decision tree, this is not identical to the tree hierarchy. (bottom): ROC-plot for sensitivity and specificity for the continuous outcome probabilities given by the decision tree. The optimal cut-off is selected to optimize both by computing the maximum distance from the diagonal.

FIG. 5 A Decision tree of 16 markers with hierarchical definition of the selected marker and cut-of value for step-wise optimization of classification. Lower boxes represent the final classification of the respective branch of the decision tree with assigned diagnosis, relative frequency of correct classification, and % of total population assigned to that branch.
B (top): Importance of biomarkers for the final classification: as each marker can be used more than once in the decision tree, this is not identical to the tree hierarchy. (bottom): ROC-plot for sensitivity and specificity for the continuous outcome probabilities given by the decision tree. The optimal cut-off is selected to optimize both by computing the maximum distance from the diagonal.

FIG. 6 A Decision tree of 15 markers with hierarchical definition of the selected marker and cut-of value for step-wise optimization of classification. Lower boxes represent the final classification of the respective branch of the decision tree with assigned diagnosis, relative frequency of correct classification, and % of total population assigned to that branch.
B (top): Importance of biomarkers for the final classification: as each marker can be used more than once in the decision tree, this is not identical to the tree hierarchy. (bottom): ROC-plot for sensitivity and specificity for the continuous outcome probabilities given by the decision tree. The optimal cut-off is selected to optimize both by computing the maximum distance from the diagonal.

FIG. 7 A Decision tree of 14 markers with hierarchical definition of the selected marker and cut-of value for step-wise optimization of classification. Lower boxes represent the final classification of the respective branch of the decision tree with assigned diagnosis, relative frequency of correct classification, and % of total population assigned to that branch.
B (top): Importance of biomarkers for the final classification: as each marker can be used more than once in the decision tree, this is not identical to the tree hierarchy. (bottom): ROC-plot for sensitivity and specificity for the continuous outcome probabilities given by the decision tree. The optimal cut-off is selected to optimize both by computing the maximum distance from the diagonal.

FIG. 8 A Decision tree of 13 markers with hierarchical definition of the selected marker and cut-of value for step-wise optimization of classification. Lower boxes represent the final classification of the respective branch of the decision tree with assigned diagnosis, relative frequency of correct classification, and % of total population assigned to that branch.
B (top): Importance of biomarkers for the final classification: as each marker can be used more than once in the decision tree, this is not identical to the tree hierarchy. (bottom): ROC-plot for sensitivity and specificity for the continuous outcome probabilities given by the decision tree. The optimal cut-off is selected to optimize both by computing the maximum distance from the diagonal.

FIG. 9 A Decision tree of 12 markers with hierarchical definition of the selected marker and cut-of value for step-wise optimization of classification. Lower boxes represent the final classification of the respective branch of the decision tree with assigned diagnosis, relative frequency of correct classification, and % of total population assigned to that branch.
B (top): Importance of biomarkers for the final classification: as each marker can be used more than once in the decision tree, this is not identical to the tree hierarchy. (bottom): ROC-plot for sensitivity and specificity for the continuous outcome probabilities given by the decision tree. The optimal cut-off is selected to optimize both by computing the maximum distance from the diagonal.

FIG. 10 A Decision tree of 11 markers with hierarchical definition of the selected marker and cut-of value for step-wise optimization of classification. Lower boxes represent the final classification of the respective branch of the decision tree with assigned diagnosis, relative frequency of correct classification, and % of total population assigned to that branch.
B (top): Importance of biomarkers for the final classification: as each marker can be used more than once in the decision tree, this is not identical to the tree hierarchy. (bottom): ROC-plot for sensitivity and specificity for the continuous outcome probabilities given by the decision tree. The optimal cut-off is selected to optimize both by computing the maximum distance from the diagonal.

FIG. 11 A Decision tree of 10 markers with hierarchical definition of the selected marker and cut-of value for step-wise optimization of classification. Lower boxes represent the final classification of the respective branch of the decision tree with assigned diagnosis, relative frequency of correct classification, and % of total population assigned to that branch.
B (top): Importance of biomarkers for the final classification; as each marker can be used more than once in the decision tree, this is not identical to the tree hierarchy. (bottom): ROC-plot for sensitivity and specificity for the continuous outcome probabilities given by the decision tree. The optimal cut-off is selected to optimize both by computing the maximum distance from the diagonal.

FIG. 12 A Decision tree of 9 markers with hierarchical definition of the selected marker and cut-of value for step-wise optimization of classification. Lower boxes represent the final classification of the respective branch of the decision tree with assigned diagnosis, relative frequency of correct classification, and % of total population assigned to that branch.
B (top): Importance of biomarkers for the final classification: as each marker can be used more than once in the decision tree, this is not identical to the tree hierarchy. (bottom): ROC-plot for sensitivity and specificity for the continuous outcome probabilities given by the decision tree. The optimal cut-off is selected to optimize both by computing the maximum distance from the diagonal.

FIG. 13 A Decision tree of 8 markers with hierarchical definition of the selected marker and cut-of value for step-wise optimization of classification. Lower boxes represent the final classification of the respective branch of the decision tree with assigned diagnosis, relative frequency of correct classification, and % of total population assigned to that branch.
B (top): Importance of biomarkers for the final classification: as each marker can be used more than once in the decision tree, this is not identical to the tree hierarchy. (bottom): ROC-plot for sensitivity and specificity for the continuous outcome probabilities given by the decision tree. The optimal cut-off is selected to optimize both by computing the maximum distance from the diagonal.

FIG. 14 A Decision tree of 7 markers with hierarchical definition of the selected marker and cut-of value for step-wise optimization of classification. Lower boxes represent the final classification of the respective branch of the decision tree with assigned diagnosis, relative frequency of correct classification, and % of total population assigned to that branch.
B (top): Importance of biomarkers for the final classification: as each marker can be used more than once in the decision tree, this is not identical to the tree hierarchy. (bottom): ROC-plot for sensitivity and specificity for the continuous outcome probabilities given by the decision tree. The optimal cut-off is selected to optimize both by computing the maximum distance from the diagonal.

FIG. 15 A Decision tree of 6 markers with hierarchical definition of the selected marker and cut-of value for step-wise optimization of classification. Lower boxes represent the final classification of the respective branch of the decision tree with assigned diagnosis, relative frequency of correct classification, and % of total population assigned to that branch.
B (top): Importance of biomarkers for the final classification: as each marker can be used more than once in the decision tree, this is not identical to the tree hierarchy. (bottom): ROC-plot for sensitivity and specificity for the continuous outcome probabilities given by the decision tree. The optimal cut-off is selected to optimize both by computing the maximum distance from the diagonal.

FIG. 16 A Decision tree of 5 markers with hierarchical definition of the selected marker and cut-of value for step-wise optimization of classification. Lower boxes represent the final classification of the respective branch of the decision tree with assigned diagnosis, relative frequency of correct classification, and % of total population assigned to that branch.
B (top): Importance of biomarkers for the final classification: as each marker can be used more than once in the decision tree, this is not identical to the tree hierarchy. (bottom): ROC-plot for sensitivity and specificity for the continuous outcome probabilities given by the decision tree. The optimal cut-off is selected to optimize both by computing the maximum distance from the diagonal.

FIG. 17 Accuracy (% of correct classifications) for all possible combinations of subsamples of biomarkers. Red line shows median accuracy for a given number of biomarkers.

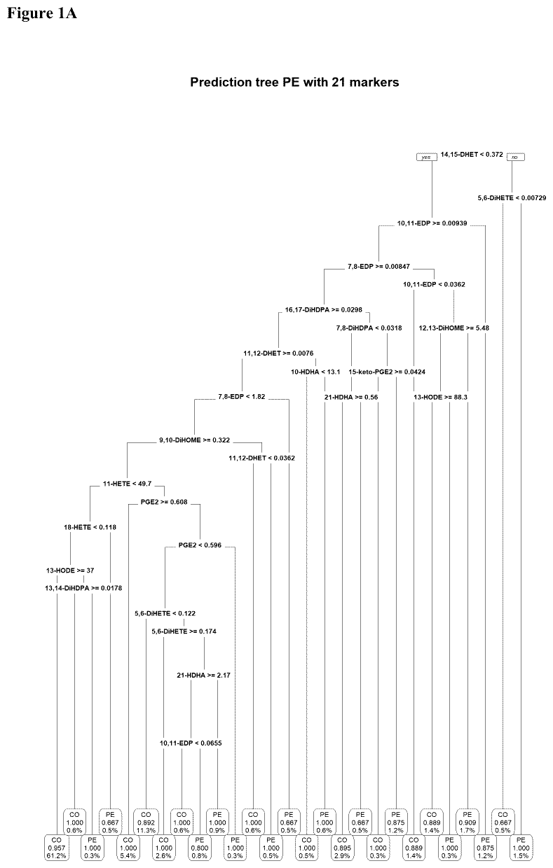

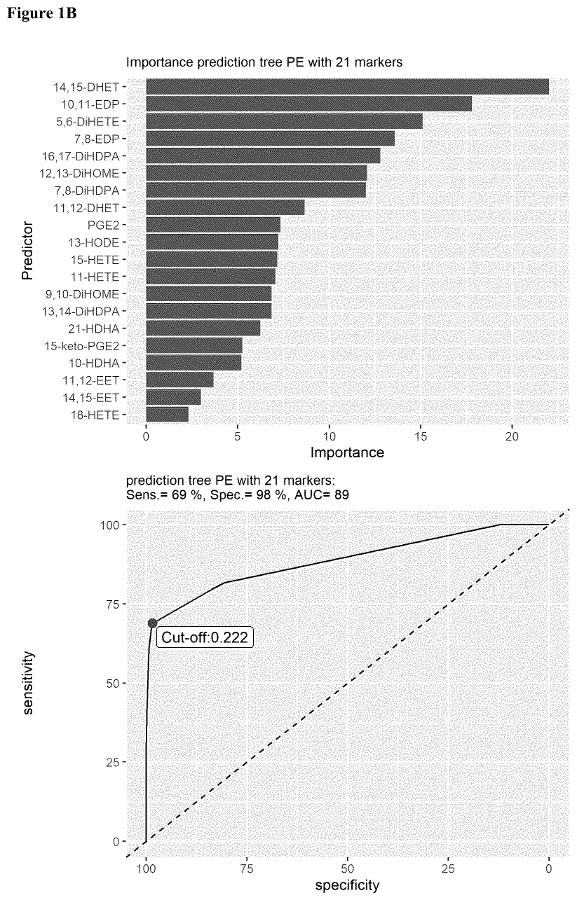

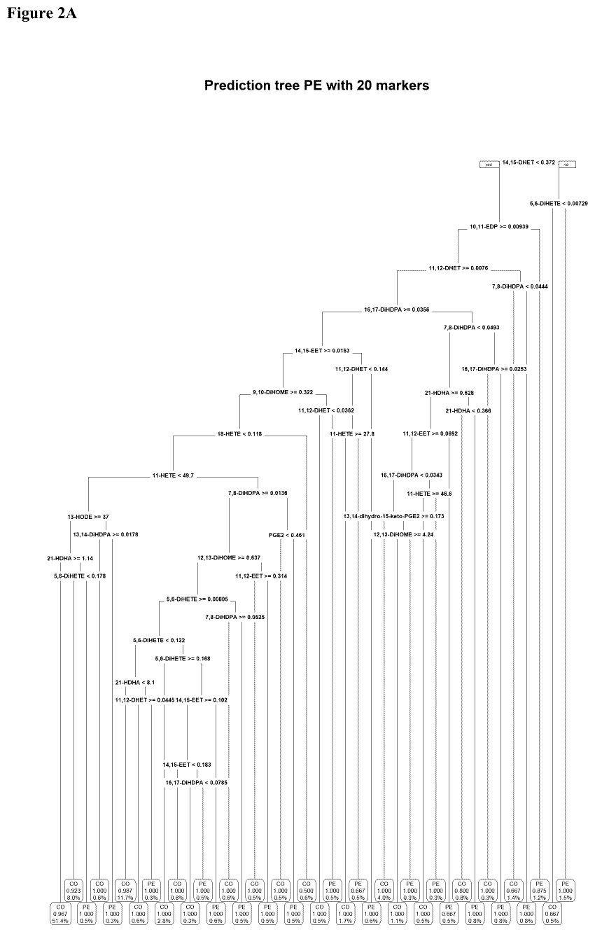

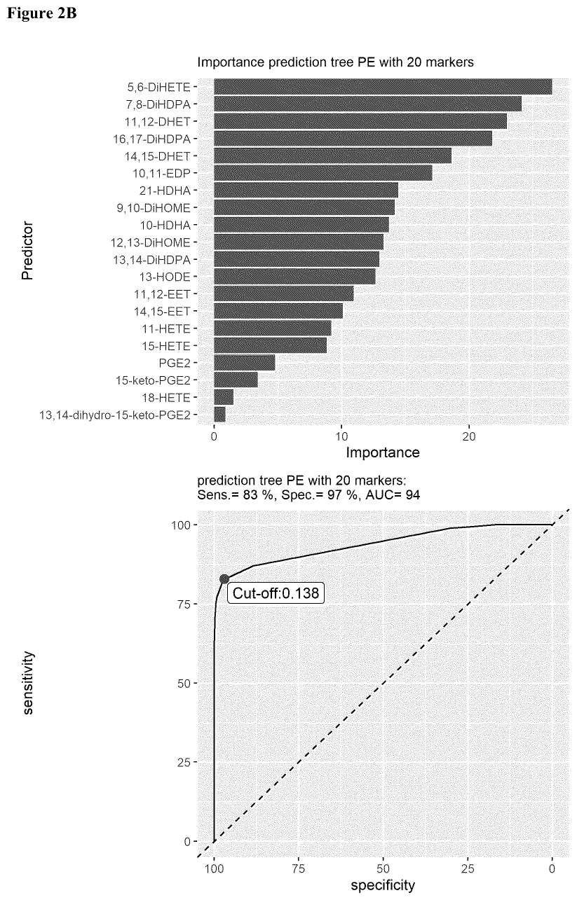

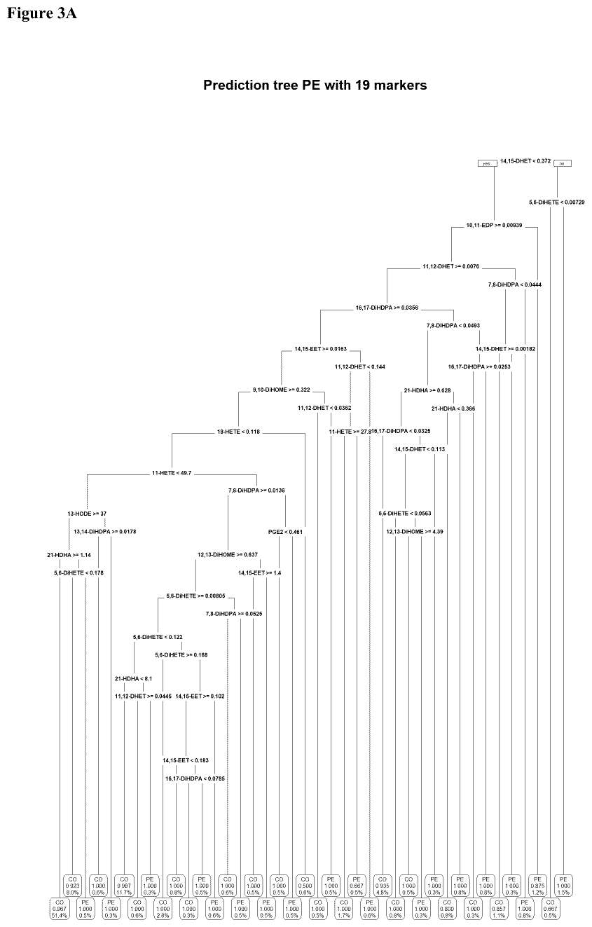

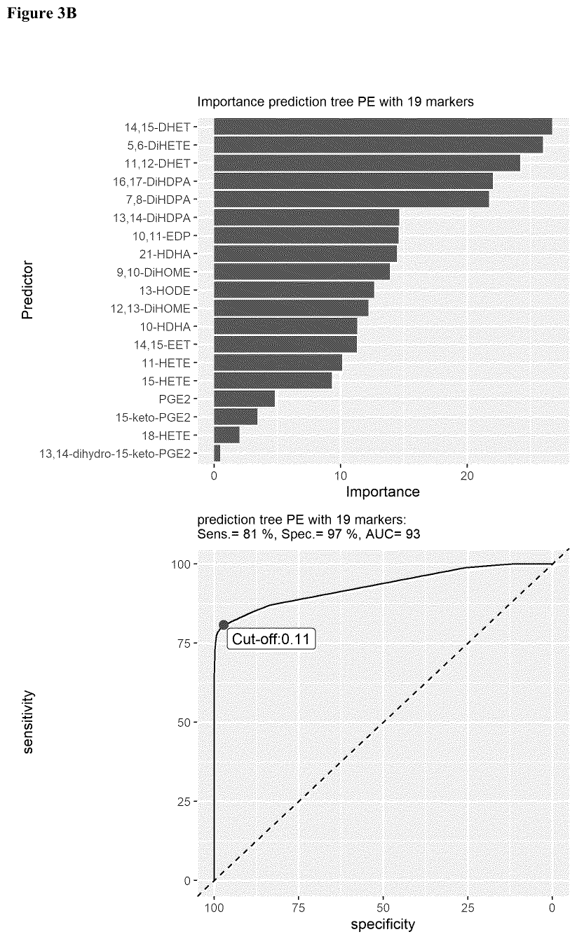

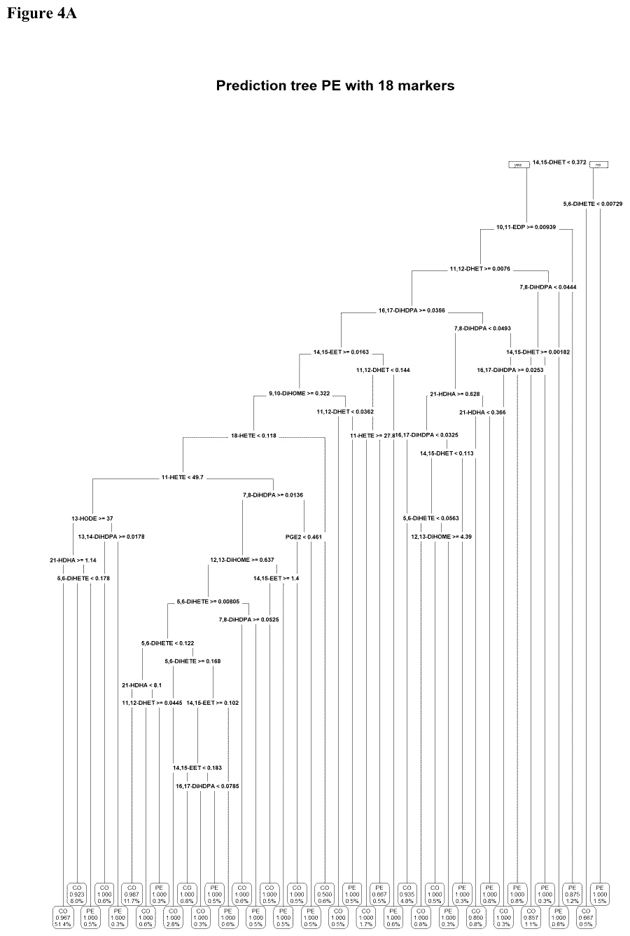

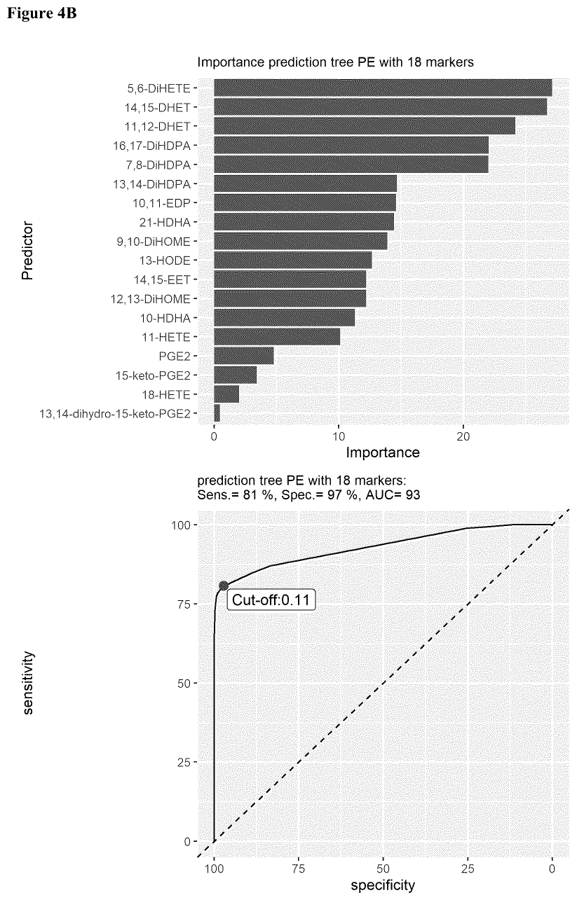

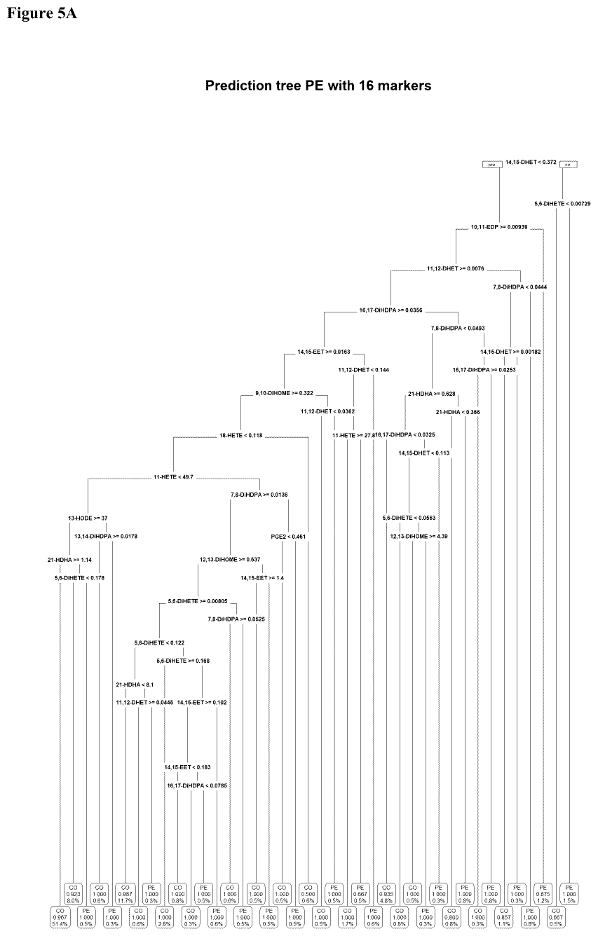

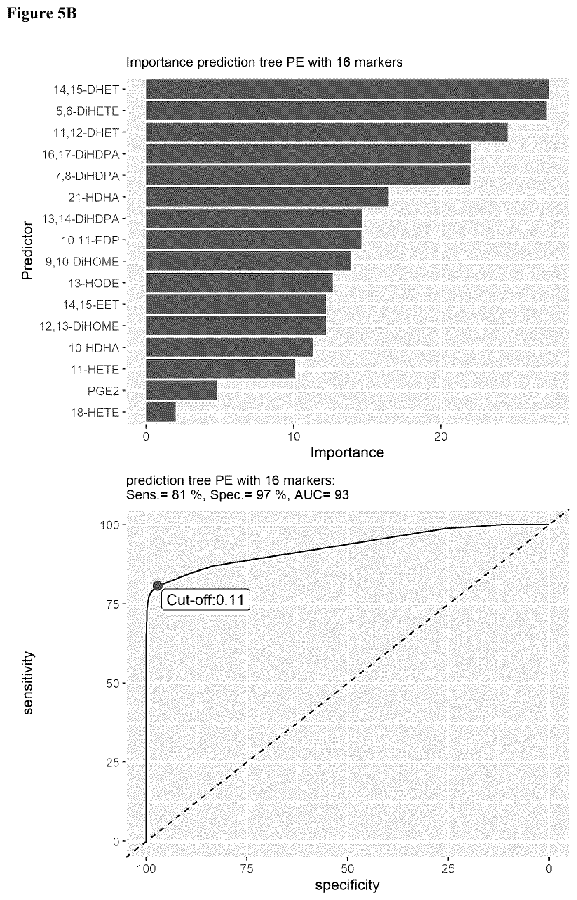

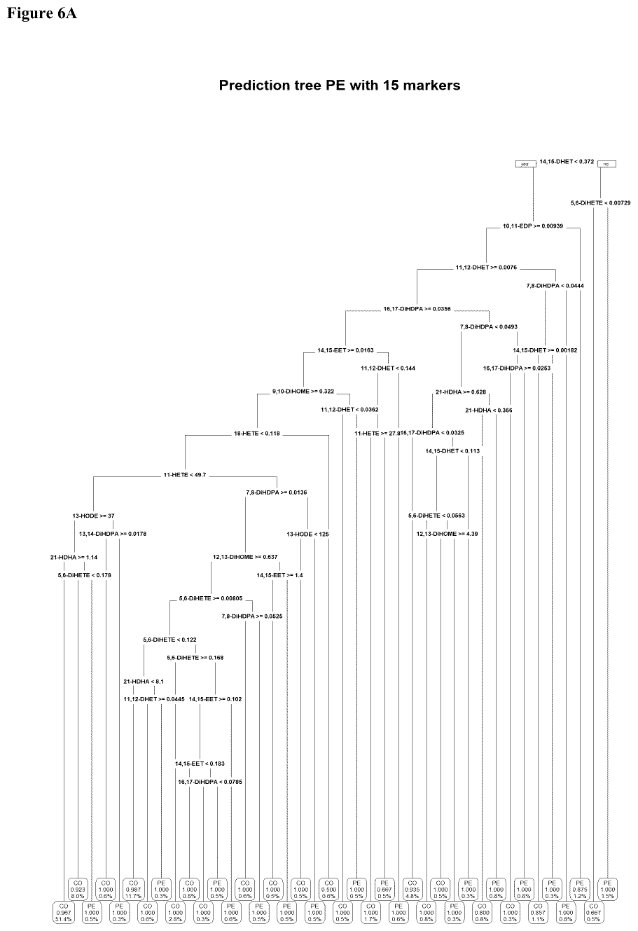

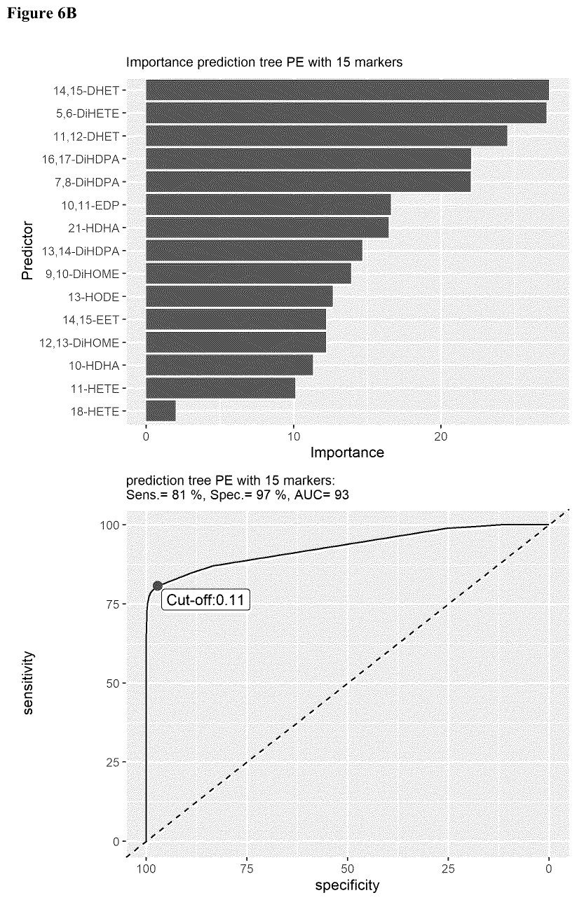

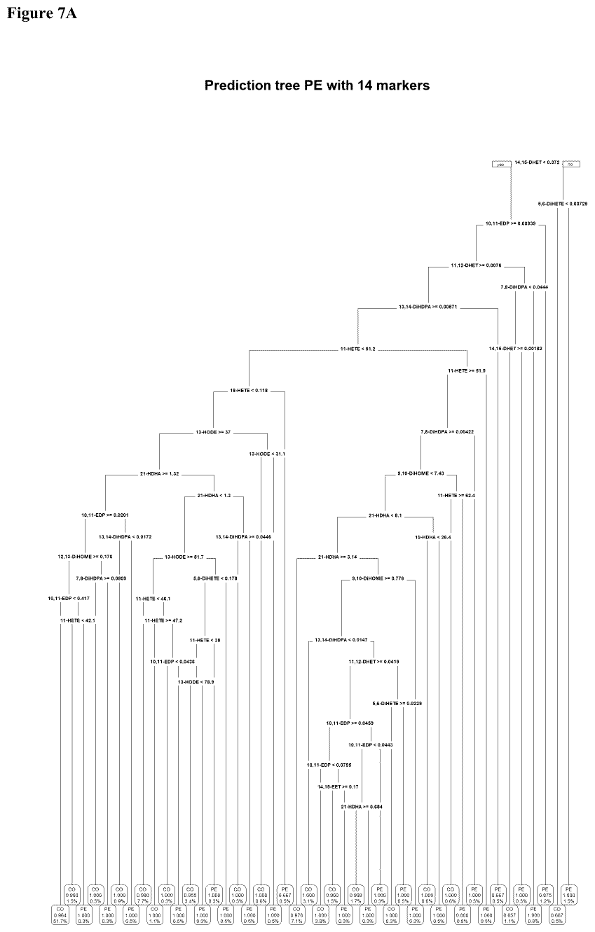

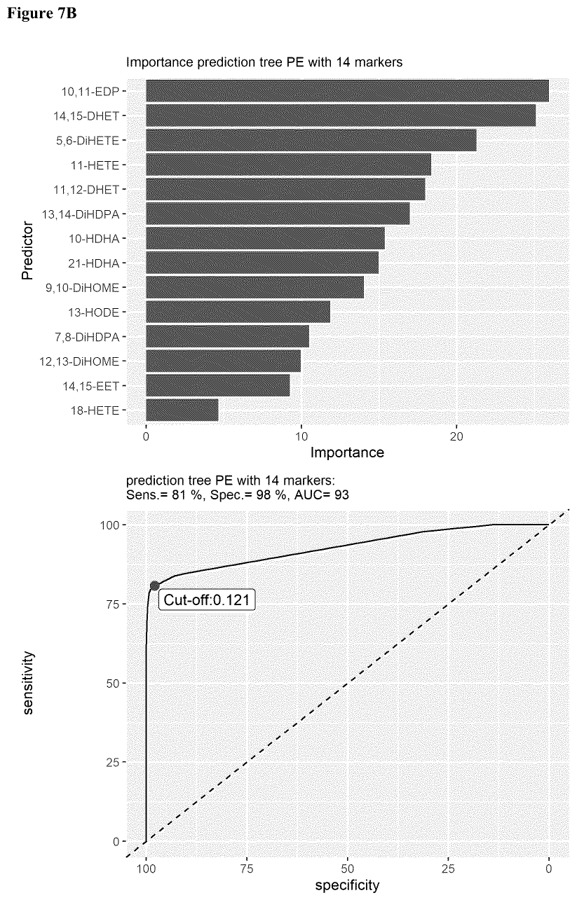

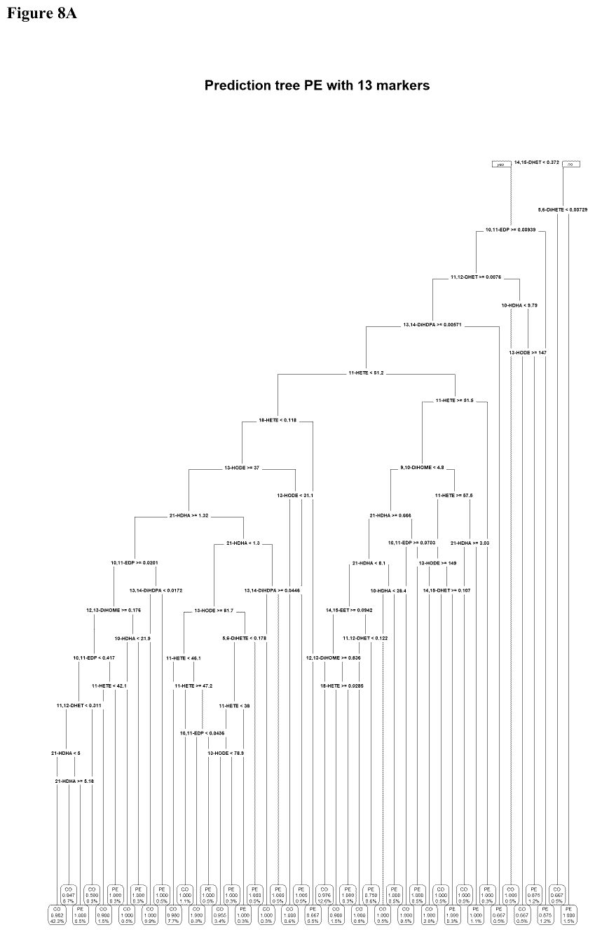

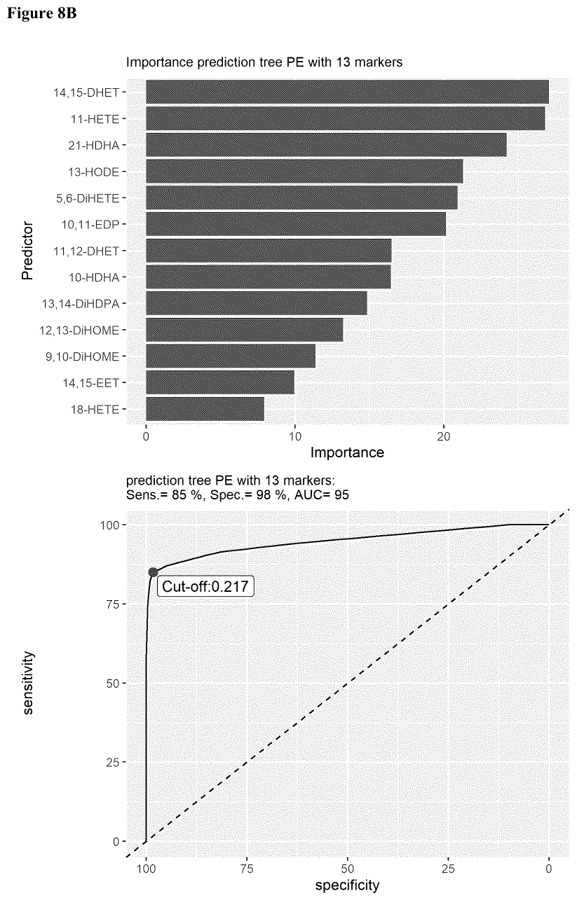

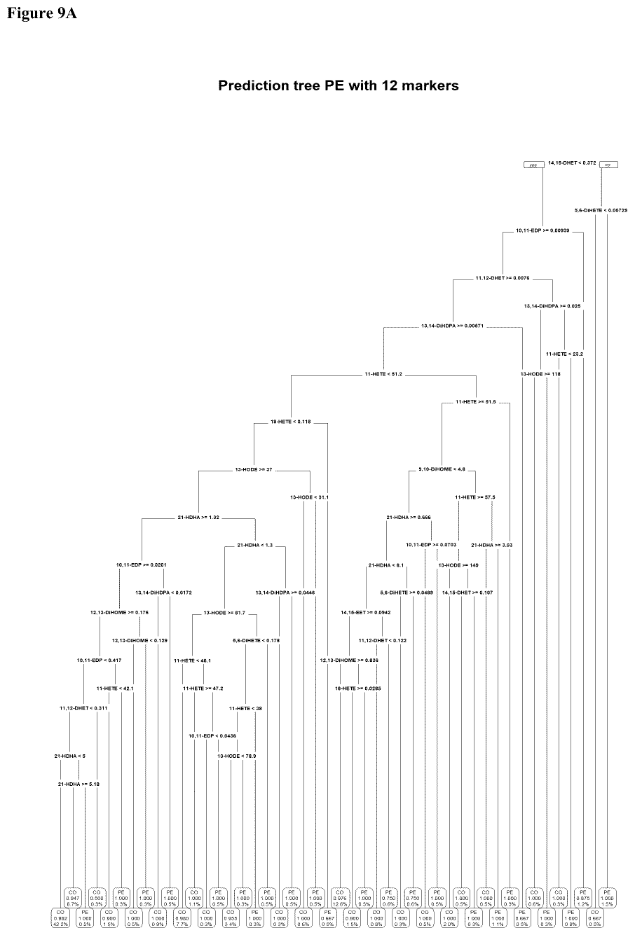

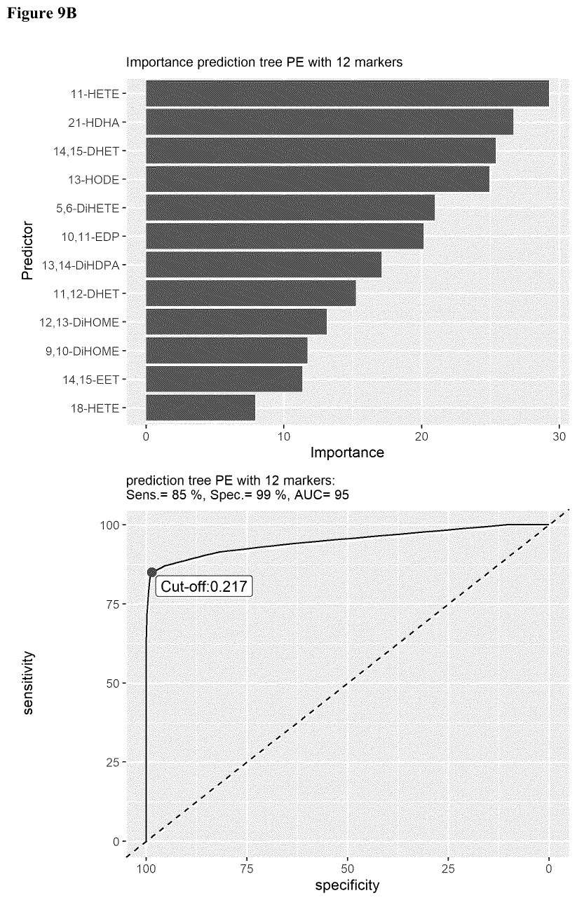

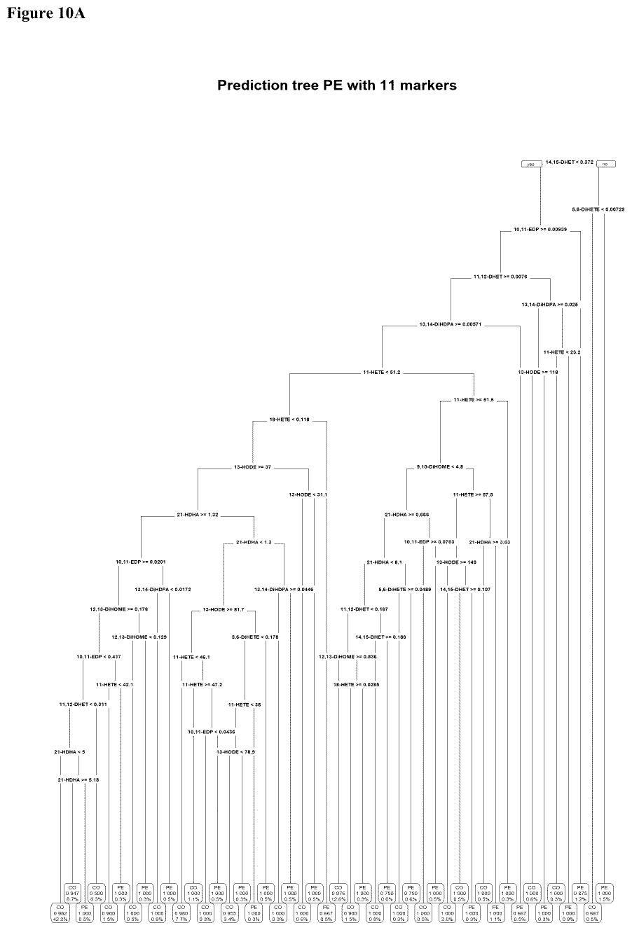

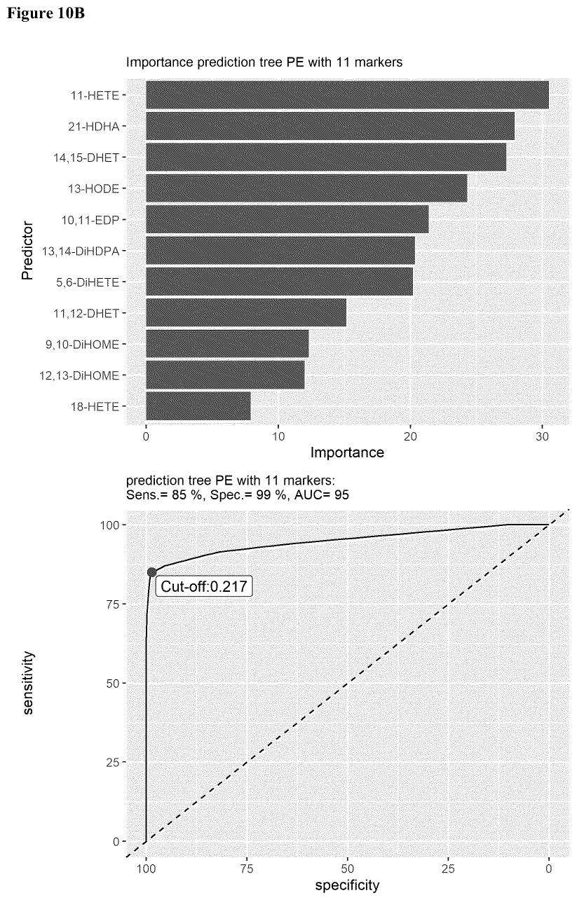

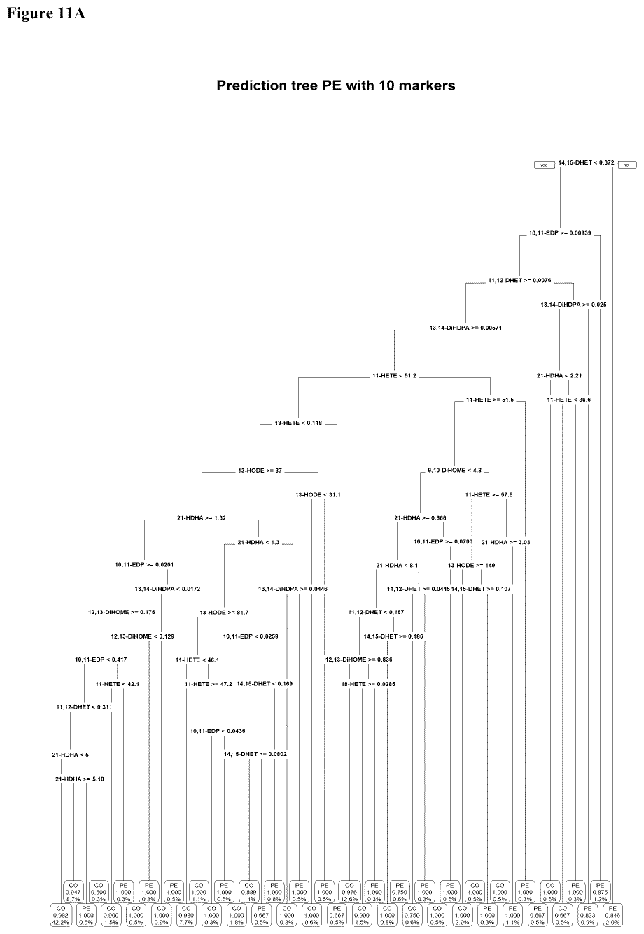

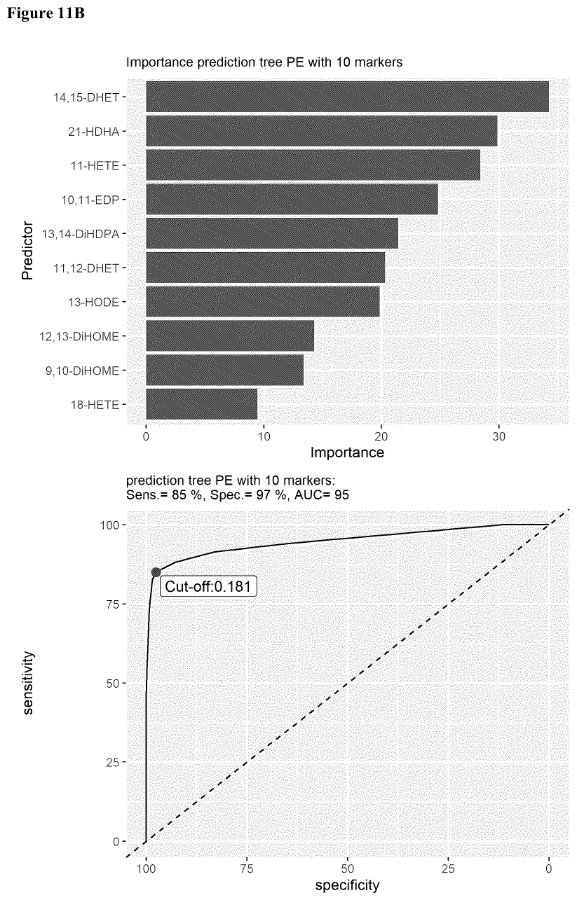

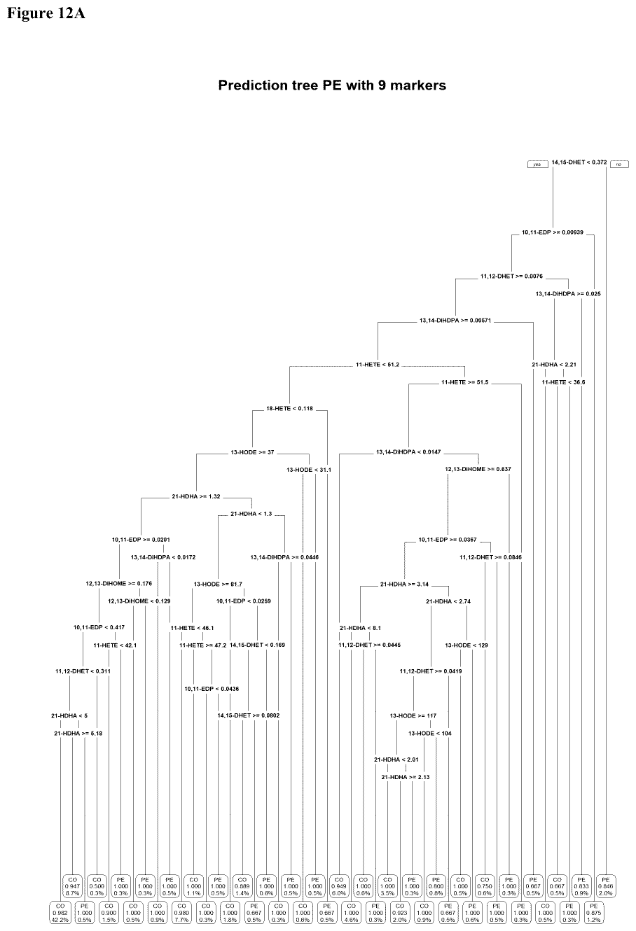

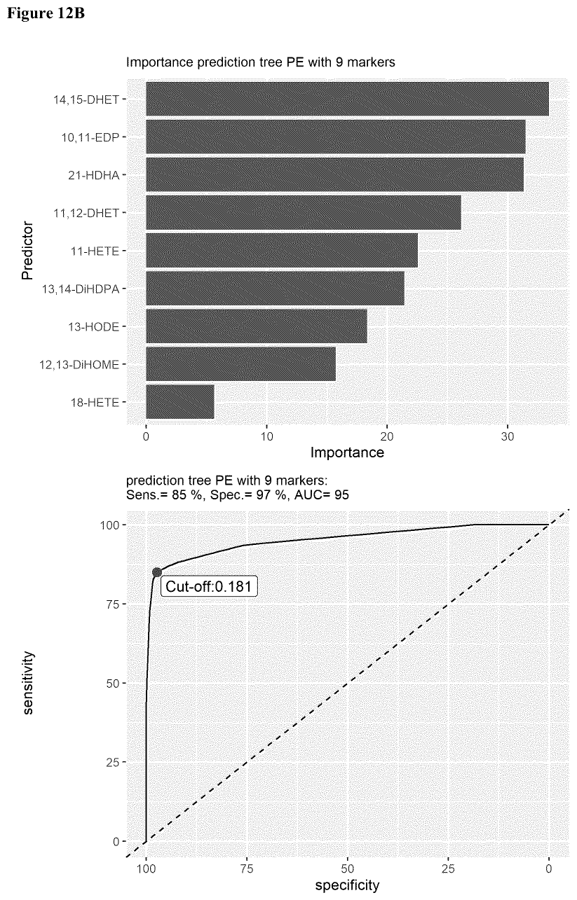

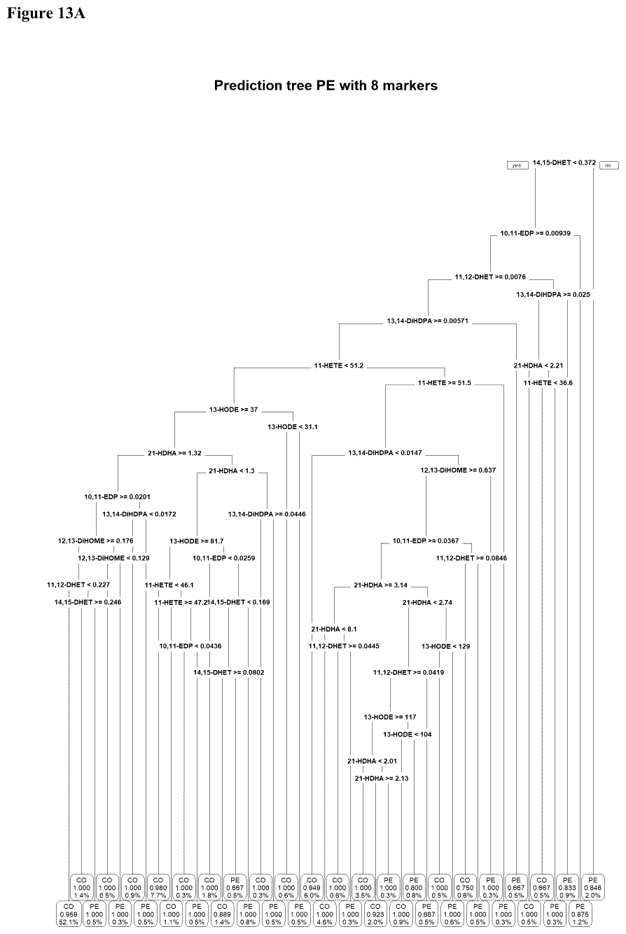

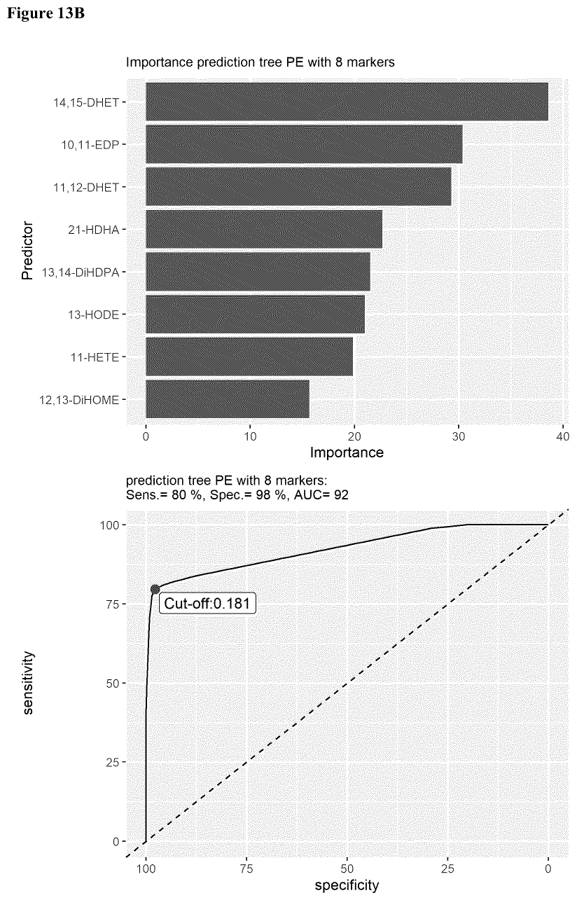

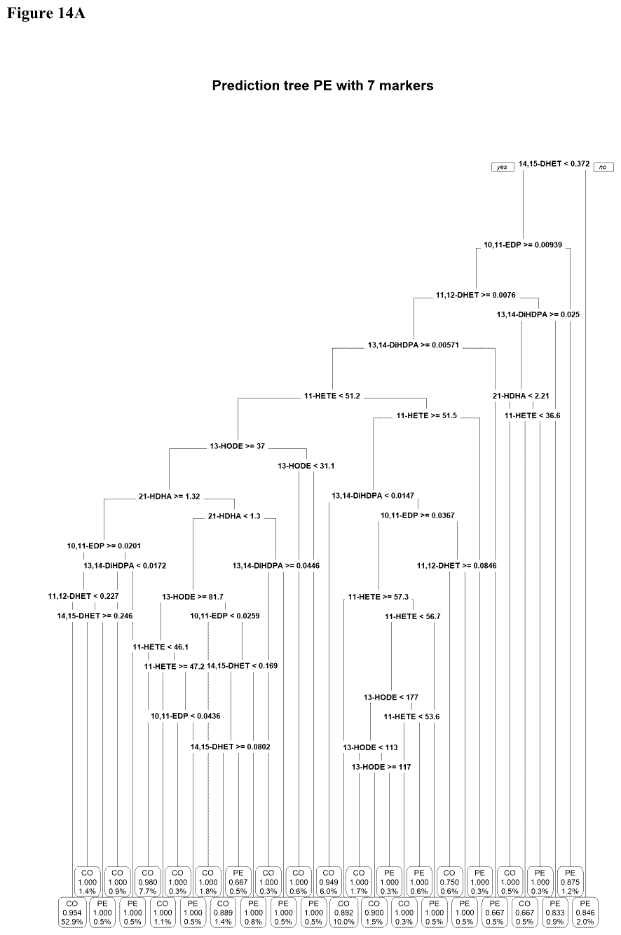

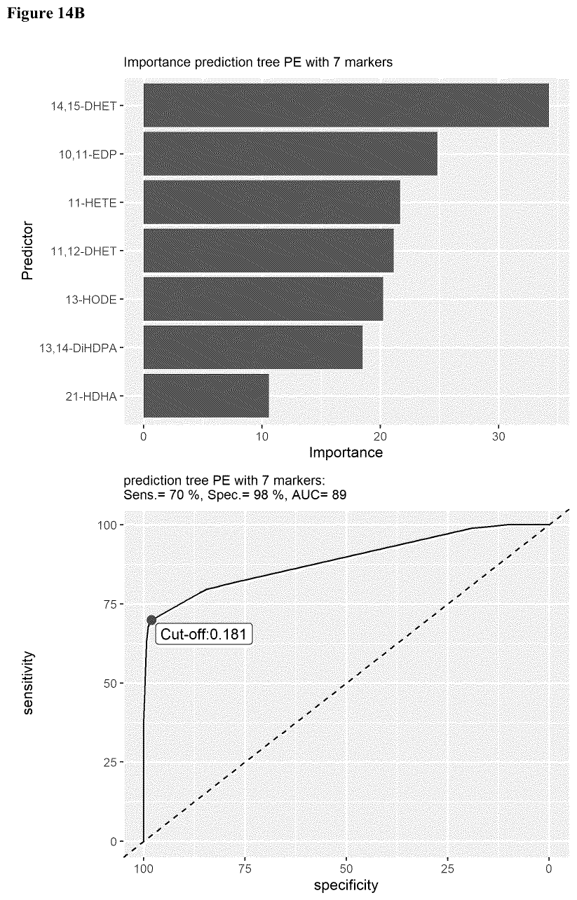

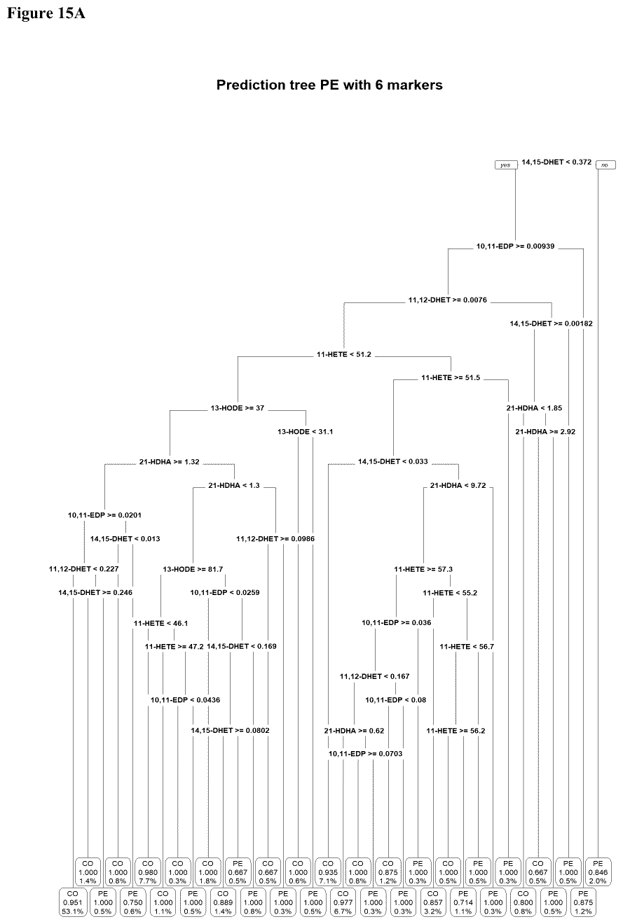

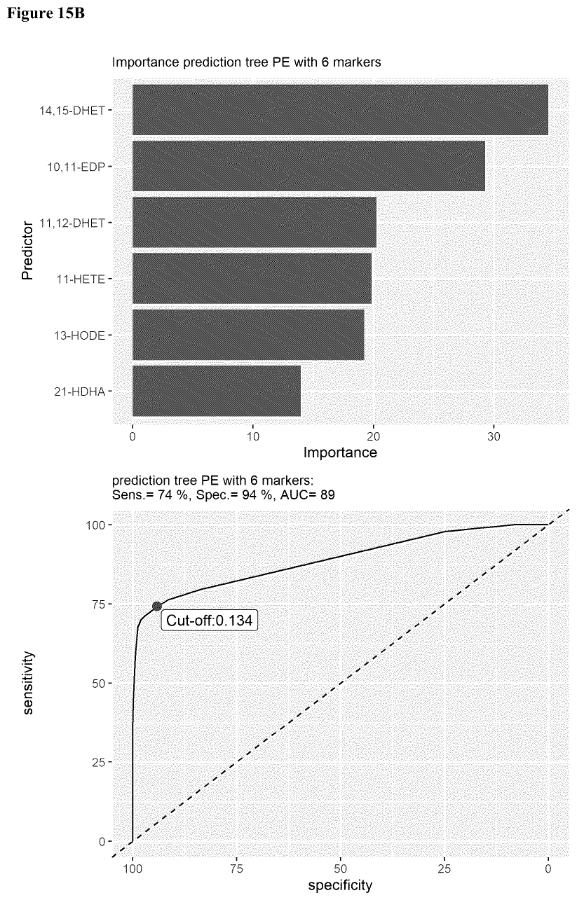

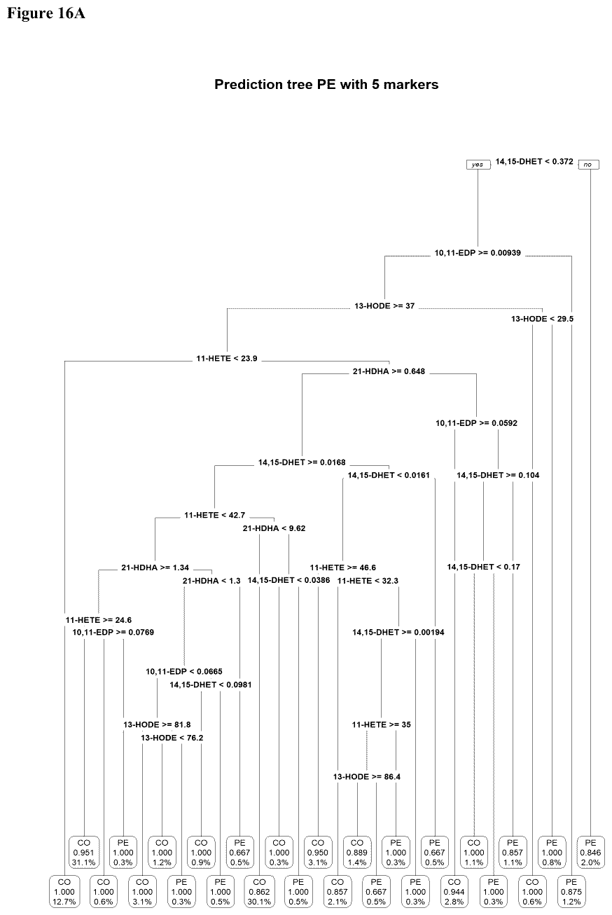

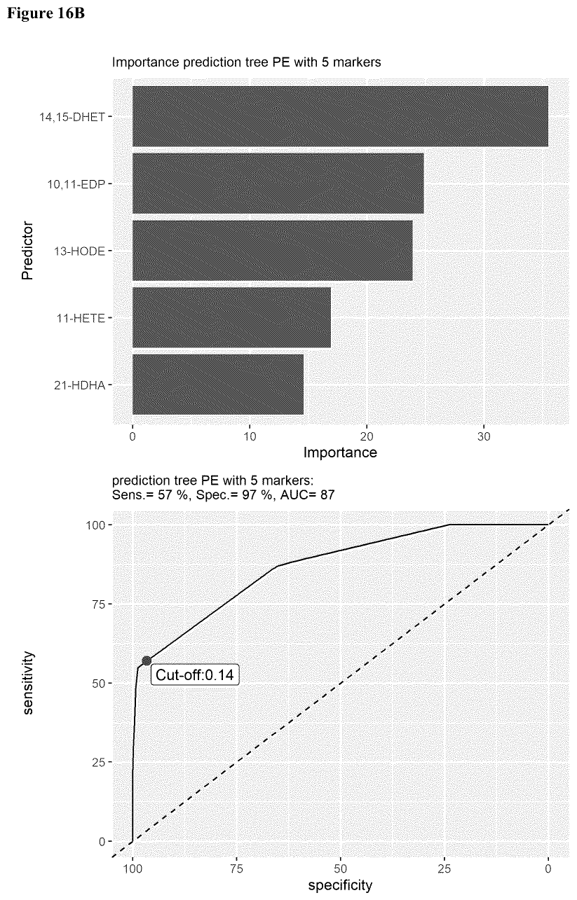

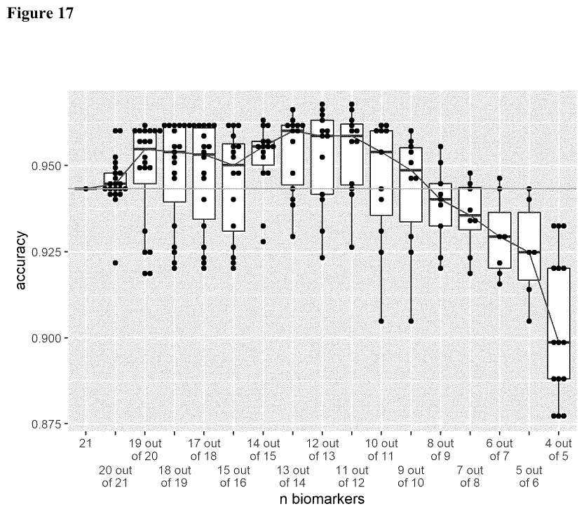

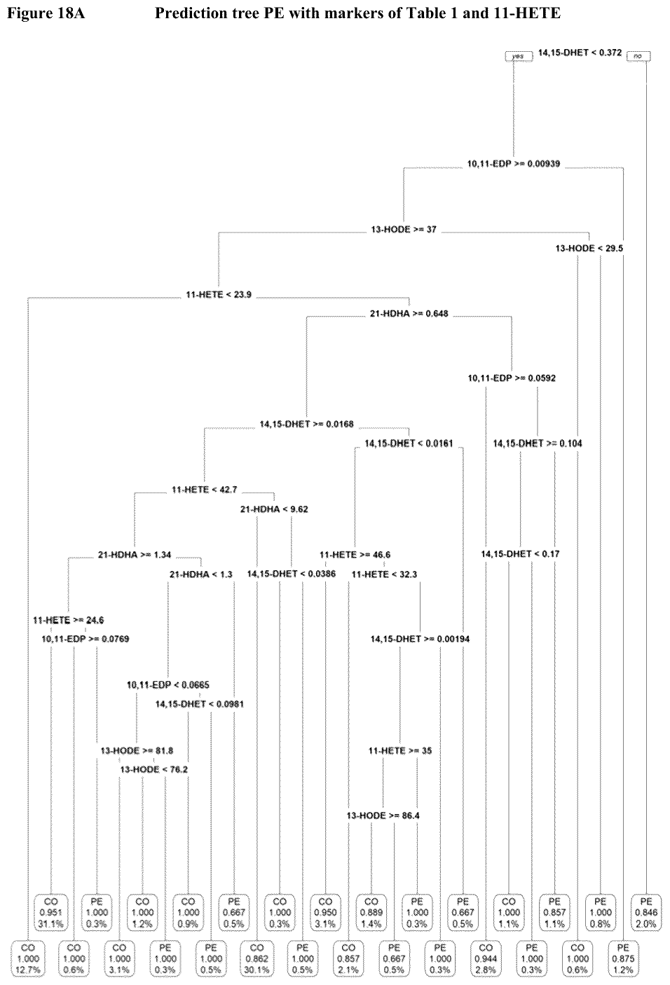

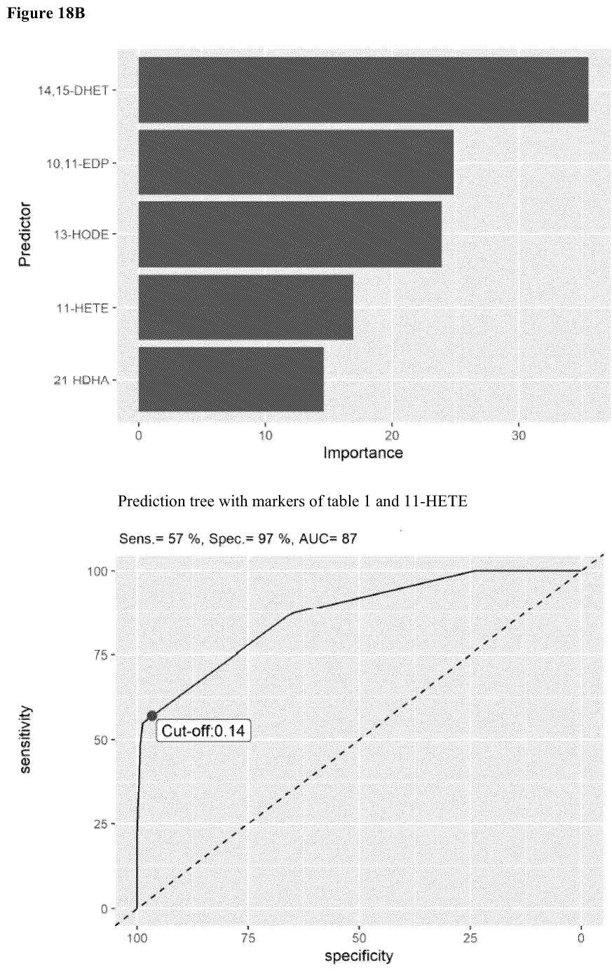

FIG. 18 Decision tree with markers of table 1 and 11-HETE with hierarchical definition of the selected marker and cut-of value for step-wise optimization of classification. Lower boxes represent the final classification of the respective branch of the decision tree with assigned diagnosis, relative frequency of correct classification, and % of total population assigned to that branch.
B (top): Importance of biomarkers for the final classification; as each marker can be used more than once in the decision tree, this is not identical to the tree hierarchy. (bottom): ROC-plot for sensitivity and specificity for the continuous outcome probabilities given by the decision tree. The optimal cut-off is selected to optimize both by computing the maximum distance from the diagonal.

1. A method for the prediction of preeclampsia in a pregnant female human subject until the end of gestational week 16 of pregnancy based on metabolites from a blood sample obtained from said subject, comprising:
measuring the level of the following metabolites in the sample: 14,15-DHET, 13-HODE, 10,11-EDP, 21-HDHA, and 11,12-DHET, and
concluding based on the measured level whether the subject is likely to develop preeclampsia.

2. The method of claim 1, further comprising:
measuring the level of at least one of the following metabolites in the sample:
7,8-EDP, 16,17-DiHDPA, 12,13-DiHOME, 11,12-DHET, 9,10-DiHOME, 5,6-DiHETE; 10-HDHA, 11,12-EET, 15-HETE, 7,8-DiHDPA, PGE2, 14,15-EET, 18-HETE, EODAcis, EODAtrans, and 13,14-DiHDPA.

3. The method of claim 1, further comprising:
measuring the level of 11-HETE.

4. The method of claim 1, wherein the level of the following metabolites in the sample is measured: 14,15-DHET, 13-HODE, 10,11-EDP, 21-HDHA, and 11-HETE;
5,6-DiHETE; 11,12-DHET; 13,14-DiHDPA; 12,13-DiHOME; 9,10-DiHOME; and 18-HETE.

5. The method of claim 1, wherein the level of the following metabolites in the sample is measured: 14,15-DHET, 13-HODE, 10,11-EDP, 21-HDHA, and 11-HETE;
5,6-DiHETE; 11,12-DHET; 13,14-DiHDPA; 12,13-DiHOME; 9,10-DiHOME; 18-HETE; and 14,15-EET.

6. The method of claim 1, wherein the level of sFLT-1 and/or PlGF is measured.

7. The method of claim 1, wherein the method is performed within the first trimester of pregnancy.

8. The method of claim 1, wherein the measuring of metabolite levels is performed using a mass spectrometer.

9. The method of claim 1, wherein the decision whether the subject has preeclampsia comprises the step of training a classification algorithm on a training set of cases and controls, and applying it to measured metabolite levels, wherein the classification method is a random forest method, a support vector machine (SVM), or a K-nearest neighbor method (K-NN).

10. (canceled)

11. A kit for the prediction of preeclampsia in a female human subject based on metabolites obtained from a blood sample, comprising samples of the following metabolites:
14,15-DHET, 13-HODE, 10,11-EDP, 21-HDHA, and 11,12-DHET, in particular for use as a standard in mass spectrometry.

12. The kit of claim 11, wherein the sample carries a marker that allows for the differentiation from the naturally occurring metabolite in mass spectrometry, in particular wherein the marker is deuterium.

13. The kit of claim 11, further comprising
Instructions for treating the development of preeclampsia,
a means or reagent for extracting eicosanoids, in particular a separation column,
a chromatography column, and/or
a quality control specimen.

14. (canceled)

15. A method for preparing a metabolite profile that is indicative of the presence or absence of preeclampsia in a subject, comprising:
isolating metabolites from a blood sample obtained from the subject, and
measuring the level of the following metabolites in the sample: 14,15-DHET, 13-HODE, 10,11-EDP, 21-HDHA, and 11,12-DHET.

16. (canceled)
